(19) United States
(12) Patent Application Publication
(10) Pub. No.: US 2008/0199519 A1
(43) Pub. Date: Aug. 21, 2008

(54) Production of Double-or Multi-Layered Microcapsules

(75) Inventors: Eric Thoenes, Buedingen (DE); Peter Geigle, Alzenau (DE)

Correspondence Address: NIXON & VANDERHYE, PC, 901 NORTH GLEBE ROAD, 11TH FLOOR, ARLINGTON, VA, 22203, US

(73) Assignee: CELLMED AG, Alzenau (DE)

(21) Appl. No.: 11/719,629

(22) PCT Filed: Sep. 22, 2005
(86) PCT No.: PCT/EP05/10277

§ 371 Date: May 17, 2007

(30) Foreign Application Priority Data
Nov. 18, 2004 (DE) 10 2004 055 729.2

(51) Int. Cl. A61K 9/48; A61K 38/00

(52) U.S. Cl. 424/463; 514/12

(57) ABSTRACT

The invention relates to a method for production of double- or multi-layered micro-capsules, comprising an inner microcapsule of cross-linked polymers and biological cells and one or more layers of cross-linked polymers without biological cells, which completely enclose(s) the inner microcapsule, whereby, in a first method step, single-layered microcapsules of cross-linked polymers with biological cells are produced and, in at least one further method step, at least one outer layer shell of cross-linked polymer is applied which contains no biological cells.

### FIELD OF THE INVENTION

The invention relates to the production of polymeric microcapsules containing biological cells, characterised in that the capsule has at least a double-layer structure comprising an inner core made of a crosslinked polymer with a high concentration of biological cells, and an outer covering layer made of a polymer without any biological cells.

The production method is structured in two steps. In a first step a mixture of the biological cells to be encapsulated and a soluble form of the polymer is pushed through an air-operated spray nozzle comprising at least two channels such that drops are produced which fall into a coagulation bath containing crosslinking agent. In this way spherical balls with a single-layer structure are produced which contain biological cells. In a second step, these single-layer balls are absorbed once again in a solution of polymers without any biological cells and are dropped again by means of the air-operated spray nozzle comprising three channels and crosslinked in the coagulation bath. In this way a coating layer without any biological cells is applied to the single-layer balls, and balls with a double-layer structure are produced. The second step can be repeated several times so that capsules with a multiple-layer structure are produced.

### BACKGROUND TO THE INVENTION

In medicine it is known with allogenic or xenogenic transplants that immune reactions of the host organism can be countered by micro-encapsulation of the transplant (biological cells or biological tissue) in polymers in order to immunoisolate the latter (Lim F and Sun A M in “Science”, vol. 210, 1980, pages 908-910; Stevenson W T K and Sefton M V. 1992, in “Fundamentals of Animal Cell Encapsulation and Immobilization”, MFA Goosen, ed., CRC Press, Boca Raton, Fla.).

With these methods exogenous biological cells (if appropriate genetically modified) or exogenous biological tissue is encapsulated (immobilised) in a matrix, preferably made of polymers, by means of a wide variety of dropping methods and transplanted into the patient. This matrix must on the one hand be permeable for the supply of oxygen and nutrients to the cells, and on the other hand must enable the diffusion of the therapeutic protein out of the capsule into the patient. On the other hand, components of the body's own immune system must not pass through the matrix. Synthetic, semi-synthetic and natural, water-soluble biopolymers (e.g. alginates, hyaluronic acid, cellulose sulphates etc.) can be used here as matrix material. Due to its bio-compatibility and its cross-linking properties alginates are ideally suitable for this application. From a chemical viewpoint, alginates are anionic polysaccharides from homopolymeric groups of β-D-mannuronic acid and α-L-guluronic acid, separated by heteropolymeric regions of both acids. In the presence of monovalent cations, such as for example sodium or potassium, alginates are water-soluble and form highly viscous solutions. By the interaction of the individual alginate chains with di-, tri- or multivalent cations (such as calcium, barium or polylysine), a cross-linked non-water-soluble hydrogel is produced.

When producing the spherical microcapsules it is of crucial significance that the encapsulated cells or tissue are/is fully embedded in the polymer matrix. Cells/tissue lying very close to the edge or cells or tissue parts projecting from the matrix would disable the principle of immunoisolation because the immune defence would recognise these exogenous components and destroy the transplant. With single-layer microcapsules this can only be avoided if the concentration of the cells in the suspension to be dropped is kept very low in order to avoid the probability of cells positioned at the edge. In order to achieve the highest level of active agent, however, and to keep the overall transplant volume as low as possible, it is mostly necessary to encapsulate very high concentrations of biological cells. Therefore, it is advantageous to apply a further covering layer made of a pure polymer around the single-layer microcapsule. In the literature, so-called three-channel nozzles are suggested for this (Jork et al. in “Appl. Microbiol. Biotechnol.” vol. 53, 2000, pages 224-229, U.S. Ser. No. 09/762,850). The polymer/cell mixture is pushed through the inner channel here, the pure polymer without any biological cells through the second channel, and compressed air, which causes the drops to break away, through the outer channel. Thus, in one procedural step a polymer capsule with a polymer core and biological cells and an outer covering layer without any biological cells is produced. The crosslinking of the polymer in the core and in the covering layer take place at the same time. The disadvantage of this method is that due to the immediate covering, the core can not be rounded into its desired spherical form, but acquires a rather spindle-like shape with two drawn out ends. The more viscous the polymer that is used, the more this effect is emphasised and the risk of cells lying at the edge or on the surface arises due to the shape of the drawn out core. A further disadvantage with this method is that due to the spindle-shape of the inner core, the covering layer is not of uniform thickness, but is thinner (or totally non-existent) at the poles of the spindle, whereas at the equator of the capsules it is thicker. This results in different lengths of diffusion paths for molecules, e.g. for oxygen, nutrients or proteins. This can be associated with varying supply or even dying off of the encapsulated cells, or uneven rejection over time of the therapeutically effective protein produced by the encapsulated cells. Moreover, the unfavourable geometric shape also gives rise to a large ratio of overall volume to core volume. This means that a large part of the available overall volume can not be used optimally for the encapsulation of cells and so the concentration of biological cells is low in comparison to the overall volume. In order to nevertheless achieve a sufficient number of cells to be encapsulated, the overall transplant volume must unnecessarily be increased.

Another possibility known from the literature for surrounding the single-layer capsules with an additional covering layer is crosslinking with polycations, such as e.g. poly-L-lysine. According to the prior art, these single-layer microcapsules containing biological cells are made e.g. of alginate, and are incubated in a solution containing poly-L-lysine. By binding poly-L-lysine to the alginate, a so-called polyanionpolycation membrane is formed. These capsules are then immersed again in a solution of anionic polymer (e.g. alginate) which in turn binds ionically to the poly-L-lysine layer. A disadvantage, however, with the alginate-poly-L-lysine-alginate capsules is a strengthened immune reaction by the transplant recipient due to the polycation, and moreover the poly-L-lysine has proved to be cytotoxic for the encapsulated cells (King et al. in “J. Biomed Mater Res.”, vol. 57, 2001, pages 374-383; Strand et al. in “Cell Transplant”, vol. 10, 2001, pages 263-275; De Vos et al. in “Biomaterials”, vol. 18, 1997, pages 273-278). Moreover, the thickness of the outer covering layer is very small (ionically crosslinked mono-layer) when using this method and can not be set variably.

### SUMMARY OF THE INVENTION

The present invention describes a method for producing microcapsules with a double- or multiple- layer structure comprising an inner capsule made of polymers and biological cells (core) and one or more layers made of polymers without any biological cells which fully enclose the core. Advantageously, the microcapsules are produced in two or more procedural steps.

By means of this invention, a spherical core made of crosslinked biopolymer and biological cells is advantageously achieved. The core is characterised by homogeneous distribution of the biological cells and even thickness of the outer covering layer. A further advantage of the present invention is that by means of this method, there is a low ratio of overall to core volume and a high concentration of encapsulated biological cells in relation to the overall transplant volume. Advantageously, by varying the channel geometry for the core diameter and the thickness of the outer covering layer almost any variation is possible.

All of the encapsulation materials known from the prior art can be used to form the core and for the structure of the outer covering layer. Preferably, purified alginates (e.g. according to DE 198 36 960) are used. Whereas alginates with an average molar mass of from 20 kDa to 10,000 kDa can be used, the molar mass is preferably from 100 kDa to 1200 kDa. The viscosity of a 0.1% (w/v) aqueous alginate solution produced from the alginate to be used can be from 3 to 100 mPa-s, and it is preferably between 10 and 60 mPa-s. The concentration of the alginate for production of the alginate solution to be used for forming the single-layer capsules and the covering layer is between 0.1 and 4% (w/v), and preferably between 0.4 and 1% (w/v). Different alginate concentrations can be chosen for the single-layer capsules and for the covering layer.

In order to produce the proposed microcapsules, in a first procedural step the single-layer microcapsule is first of all formed from crosslinked polymer with biological cells. For this, a mixture (suspension) of the soluble form of the polymer e.g. alginate (e.g. potassium or sodium alginate in a physiological cooking salt solution) and the biological cells is first of all produced in a concentration of up to 5*10⁷ cells per ml alginate solution. In the case of encapsulating tissue particles instead of individual biological cells, the concentration can be chosen to be substantially lower.

The homogeneous cell/alginate suspension is pushed through an air-operated spray nozzle which has a three-channel structure: an inner channel, an outer nozzle and an outer air ring. For the inner channel, cannulas with an inner diameter of from 50 pm to 2000 μm are preferably used. The outer nozzle has an inner diameter of from 60 μm to 4000 μm, and the outer air ring preferably has an inner diameter of from 100 μm to 5000 μm. In the first procedural step for producing the microcapsules with a single-layer structure, only the inner channel and the outer air ring are required. For this step therefore, a spray nozzle with a corresponding structure comprising two channels can also be used. When using a three-channel spray nozzle, there is no material flow through the inner nozzle. The suspension is pushed through the inner channel at a speed of from 10 μl/min to 5 ml/min so that drops form at the bottom outlet of the cannula, and said drops break away due to the flow of air which is conveyed through the outer air ring at a speed of from 0.5 l/min to 10 l/min. The drops containing biological cells fall into a solution containing the crosslinking agent and which is located at a distance of from 4 cm to 60 cm below the bottom end of the spray nozzle. While falling, the drop rounds so as to form a spherically geometric shape. If alginates are being used, the solution containing the crosslinking agent preferably comprises bivalent cations e.g. dissolved calcium or barium (5-100 mM) or other bivalent or multivalent cations. In addition, the coagulation bath preferably also contains a buffer substance (e.g. histidine 1 mM-10 mM) and cooking salt (e.g. 290 mOsmol).

If polymers other than alginates are used, crosslinking substances and buffer substances corresponding to the prior art are to be used. The crosslinking agents bring about ionic crosslinking of the polymers, and non-water-soluble microcapsules with a single-layer structure and from 50 μm to 3000 μm in size are thus formed. The diameter of the microcapsules depends upon the chosen size and geometry of the channels used. After dropping, several steps of washing the microcapsules with a physiological cooking salt solution or another suitable washing solution and if appropriate incubation in a sodium sulphate solution, preferably according to U.S. Pat. No. 6,592,886, preferably follow. The microcapsules are preferably separated from the coagulation and washing baths using a centrifuge or other suitable methods.

In the second procedural step the single-layer microcapsules which were produced in the first procedural step are absorbed in a polymer solution, preferably a 0.1% to 4% (w/v) alginate solution. This mixture is in turn pushed through the inner channel of the air-operated spray nozzle described above at a speed of from 10 μl/min to 5 ml/min. In this procedural step a pure polymer solution without any biological cells, preferably a 0.1% to 4% (w/v) alginate solution, is pushed through the second channel of the inner nozzle at the same time, at a speed of from 10 μl/min to 5 ml/min. Double-layer drops are thus formed at the end of the nozzle, and break away due to the flow of air which is conveyed through the outer air ring at a speed of from 0.5 l/min to 10 l/min. By means of this step, a covering layer made of the polymer used is applied to the single-layer microcapsules. The speeds at which the solutions can be pushed through the inner and the outer channel can differ from one another. The polymer concentrations of the single-layer microcapsules, the polymer solution in which these microcapsules are absorbed, and the polymer concentration from which the outer covering layer is made can also differ from one another. The drops containing biological cells fall into a solution containing the crosslinking agent which is located at a distance of from 4 cm to 60 cm below the bottom end of the spray nozzle. While falling, the drops round so as to form a spherical geometric shape. If alginates have been used, the solution containing the crosslinking agent preferably comprises bivalent cations, e.g. dissolved calcium or barium (5-100 mM) or other bivalent or multivalent cations. In addition, the coagulation bath preferably also contains a buffer substance (e.g. histidine) and cooking salt (e.g. 290 mOsmol). If other polymers are used as alginates, crosslinking substances and buffer substances corresponding to the prior art are to be used. As in the first procedural step, the crosslinking agents bring about ionic crosslinking of the polymers, and non-water-soluble microcapsules with a double-layer structure and from 60 μm to 4000 μm in size are thus formed. The diameter of the microcapsules with a double-layer structure depends upon the chosen size and geometry of the channels used. After dropping, several steps of washing the microcapsules with a physiological cooking salt solution or another suitable washing solution and if appropriate incubation in a sodium sulphate solution, preferably according to U.S. Pat. No. 6,592,886, preferably follow. The microcapsules are preferably separated from the coagulation and washing baths using a centrifuge or other suitable methods.

The second step can be repeated as often as one wishes so that multiple-layered capsules can be produced.

All known polymeric biomaterials corresponding to the prior art can be used e.g. from the field of natural polymers: proteins or polymers based on proteins (e.g. collagens, albumins and others), polyamino acids (e.g. poly-α-L-lysines, poly-L-glutamic acid and others), polysaccharides and derivatives thereof (e.g. carboxymethyl cellulose, cellulose sulphate, agarose, alginates, carrageenans, hyaluronic acid, heparin and heparin-like glucosamine sulphates, dextran and its derivatives, chitosan and its derivatives). Polymeric biomaterials from the field of synthetic polymers can also be used: aliphatic polyesters (e.g. polylactide acid, polyglycol acid, polyhydroxybutyrates, and others), polyamides, polyanhydrides, polyorthoesters, polyphosphazenes, thermoplastic polyurethanes, polyvinylalcohols, polyhydroxyethyl methacrylates, polymethyl methacrylates and polytetrafluoroethylenes.

### EXAMPLES

1. Production of double-layer microcapsules with biological cells (concentration 2×10⁷ cells/ml) and a diameter of the inner microcapsule of approx. 400 μm and a covering layer with a thickness of approx. 200 μm.

The cultivated biological cells to be encapsulated are washed with PBS (PAA, Austria) and detached using trypsin/EDTA (PAA, Austria). The reaction is quickly stopped with medium (dependent upon the cell type, e.g. RPMI, PAA, Austria) and the cell suspension is centrifuged out (8 mins at 1200 rpm). The pellet is resuspended in PBS and the number of cells is determined. The desired cell quantity of 1.4×10⁷ cells is centrifuged out again (8 mins at 1200 rpm). Next, all of the PBS is sucked off and 60 μl pellet resuspended in 80 μl PBS free from any air bubbles. This cell suspension is absorbed in 560 μl of a 0.8% (w/v) potassium alginate solution (an alginate with a viscosity of approx. 40 mPa-s of a 0.1% (w/v) aqueous solution is used).

In order to mix the resuspended cells with the alginate solution, the latter is drawn up into a 1 ml syringe with a cannula and mixed homogeneously with the cells by slowly drawing up and extracting several times. A cell concentration of 2×10⁷ cells/ml is produced.

For the production of microcapsules with a single-layer structure and with a diameter of 400 μm a cannula with an inner diameter of 400 μm is used in the air-operated three-channel spray nozzle for the inner channel. The cannula is fixed in an outer nozzle with an inner diameter of 700 μm. An air ring with an opening of 1.5 mm is screwed over the two inner channels. The homogeneous cell/alginate solution mixture is dropped through the described spray nozzle. For this, the 1 ml syringe containing the mixture is placed on the inner channel by means of a luer lock. The cell/alginate solution mixture is pushed through the inner channel at a speed of 300 μl/min. The flow of air is conveyed through the outer air ring at a speed of 2.5 l/min. The microcapsules that are produced fall into a coagulation bath containing barium (20 mM BaCl, 5 mM L-histidine, 124 mM NaCl, pH 7.0±0.1, 290 mOsmol±3) which is assembled approx. 10 cm below the spray nozzle. After having remained for 5 mins in the coagulation bath containing barium, the microcapsules are respectively washed 5 times with 20 ml PBS.

500 μl of the microcapsules with a single-layer structure are then absorbed in 500 μl of a 0.8% (w/v) alginate solution (an alginate with a viscosity of approx. 40 mPa-s of a 0.1% (w/v) aqueous solution was used) and mixed homogeneously. This suspension is drawn up into a 1 ml syringe and connected to the inner channel (inner diameter: 400 μm) of the spray nozzle by means of a luer lock and pushed through the latter at a speed of 50 μl/min. A 5 ml syringe with a 0.8% alginate solution is connected to the second inner channel (inner diameter: 700 μm) by means of a luer lock and pushed through the latter at a speed of 250 μl/min. The flow of air is conveyed through the outer air ring at a speed of 2.9 l/min. The microcapsules which are produced fall into a coagulation bath containing barium (20 mM BaCl, 5 mM L-histidine, 124 mM NaCl, pH 7.0±0.1, 290 mOsmol±3) which is assembled approx. 10 cm below the spray nozzle. After having remained for 5 mins in the coagulation bath containing barium, the microcapsules are respectively washed 4 times with 20 ml PBS and once with medium. This process produces microcapsules with a double-layer structure with an overall diameter of approx. 800 μm.

2. Production of double-layered microcapsules with biological cells (concentration 2×10⁶ cells/ml) and a diameter of the inner microcapsule of approx. 1000 μm and a covering layer with a thickness of 400 μm.

The cultivated biological cells to be encapsulated are washed with PBS (PAA, Austria) and detached using trypsin/EDTA (PAA, Austria). The reaction is quickly stopped with medium (dependent upon the cell type, e.g. RPMI, PAA, Austria) and the cell suspension is centrifuged out (8 mins at 1200 rpm). The pellet is resuspended in PBS and the number of cells is determined. The desired cell quantity of 2×10⁶ cells is centrifuged out again (8 mins at 1200 rpm). Next, all of the PBS is sucked off and 50 μl pellet resuspended in 150 μl PBS free from any air bubbles. This cell suspension is absorbed in 800 μl of a 0.6% (w/v) potassium alginate solution (an alginate with a viscosity of approx. 40 mPa-s of a 0.1% (w/v) aqueous solution was used).

In order to mix the resuspended cells with the alginate solution, the latter is drawn up into a 1 ml syringe with a cannula and mixed homogeneously with the cells by slowly drawing up and extracting several times. A cell concentration of 2×10⁶ cells/ml is produced.

For the production of microcapsules with a single-layer structure and with a diameter of approx. 1000 μm a cannula with an inner diameter of 800 μm is used in the air-operated three-channel spray nozzle for the inner channel. The cannula is fixed in an outer nozzle with an inner diameter of 1200 μm. An air ring with an opening of 2.0 mm is screwed over the two inner channels. The homogeneous cell/alginate solution mixture is dropped through the described spray nozzle. For this, the 1 ml syringe containing the mixture is placed on the inner channel by means of a luer lock. The cell/alginate solution mixture is pushed through the inner channel at a speed of 200 μl/min. The flow of air is conveyed through the outer air ring at a speed of 2.4 l/min. The microcapsules that are produced fall into a coagulation bath containing barium (20 mM BaCl, 5 mM L-histidine, 124 mM NaCl, pH 7.0±0.1, 290 mOsmol±3) which is assembled approx. 10 cm below the spray nozzle. After having remained for 5 mins in the coagulation bath containing barium, the microcapsules are respectively washed 5 times with 20 ml PBS.

1000 μl of the microcapsules with a single-layer structure are then absorbed in 1000 μl of a 0.8 % (w/v) alginate solution (an alginate with a viscosity of approx. 40 mPa-s of a 0.1% (w/v) aqueous solution was used) and mixed homogeneously. This suspension is drawn up into a 2 ml syringe and connected to the inner channel (inner diameter: 1000 μm) of the spray nozzle by means of a luer lock and pushed through the latter at a speed of 200 μl/min. A 10 ml syringe with a 0.6% alginate solution is connected to the second inner channel (inner diameter: 1200 μm) by means of a luer lock and pushed through the latter at a speed of 1000 μl/min. The flow of air is conveyed through the outer air ring at a speed of 3.4 l/min. The microcapsules which are produced fall into a coagulation bath containing barium (20 mM BaCl, 5 mM L-histidine, 124 mM NaCl, pH 7.0±0.1, 290 mOsmol±3) which is assembled approx. 10 cm below the spray nozzle. After having remained for 5 mins in the coagulation bath containing barium, the microcapsules are respectively washed 4 times with 20 ml PBS and once with medium. This process produces microcapsules with a double-layer structure with an overall diameter of approx. 1800 μm.

3. Production of double-layered microcapsules with biological tissue pieces (secondary hyperplastic parathyroid gland tissue) and a diameter of the inner microcapsule of approx. 800 μm and a covering layer with a thickness of 400 μm.

The human hyperplastic parathyroid gland tissue is split up into tissue particles (diameter: approx. 700 μm) using a scalpel. The tissue particles are washed five times for two minutes respectively with 30 ml PBS respectively. Then the pieces are absorbed in a 0.5% (w/v) potassium alginate solution (an alginate with a viscosity of approx. 40 mPa-s of a 0.1% (w/v) aqueous solution was used). The washed tissue particles are suspended in the alginate solution and drawn up into a 1 ml syringe.

For the production of microcapsules with a single-layer structure and with a diameter of approx. 800 μm a cannula with an inner diameter of 700 μm is used in the air-operated three-channel spray nozzle for the inner channel. The cannula is fixed in an outer nozzle with an inner diameter of 1500 μm. An air ring with an opening of 3.0 mm is screwed over the two inner channels. The homogeneous cell/alginate solution mixture is dropped through the described spray nozzle. For this, the 1 ml syringe containing the mixture is placed on the inner channel by means of a luer lock. The cell/alginate solution mixture is pushed through the inner channel at a speed of 500 μl/min. The flow of air is conveyed through the outer air ring at a speed of 5.0 l/min. The microcapsules that are produced fall into a coagulation bath containing barium (20 mM BaCl, 5 mM L-histidine, 124 mM NaCl, pH 7.0±0.1, 290 mOsmol±3) which is assembled approx. 10 cm below the spray nozzle. After having remained for 5 mins in the coagulation bath containing barium, the microcapsules are respectively washed 5 times with 20 ml PBS.

The single-layered microcapsules containing tissue and to be found in the PBS solution are once again drawn up into a 1 ml syringe and transferred into a new petri dish. All of the PBS solution is drawn off and the microcapsules with a single-layer structure are absorbed and homogeneously mixed in 500 μl of a 0.5% (w/v) alginate solution. This suspension is drawn up into a 1 ml syringe and connected to the inner channel (inner diameter: 800 μm) of the spray nozzle by means of a luer lock and pushed through the latter at a speed of 200 μl/min. A 5 ml syringe with a 0.5% alginate solution (an alginate with a viscosity of approx. 40 mPa-s of a 0.1% (w/v) aqueous solution) is connected to the second inner channel (inner diameter: 1500 μm) by means of a luer lock and pushed through the latter at a speed of 1000 μl/min. The flow of air is conveyed through the outer air ring at a speed of 4.0 l/min. The microcapsules which are produced fall into a coagulation bath containing barium (20 mM BaCl, 5 mM L-histidine, 124 mM NaCl, pH 7.0±0.1, 290 mOsmol±3) which is assembled approx. 10 cm below the spray nozzle. After having remained for 5 mins in the coagulation bath containing barium, the microcapsules are respectively washed 4 times with 20 ml PBS and once with medium. This produces microcapsules with a double-layer structure, which contain tissue, and with an overall diameter of 1600 μm.

1. A method for producing spherical microcapsules with a double- or multiple-layer structure,
comprising an inner microcapsule made of crosslinked polymers and biological cells and one or more layers of crosslinked polymers without any biological cells which fully enclose the inner microcapsule with the biological cells using a three-channel spray nozzle with an inner channel, an outer channel and an outer air ring, in a first procedural step microcapsules with a single-layer structure being produced from crosslinked polymers with biological cells, a homogeneous cell/polymer suspension being pushed through the inner channel of the three-channel spray nozzle and in at least one further procedural step at least one outer covering layer made of crosslinked polymers and which does not contain any biological cells being applied to the microcapsules with a single-layer structure, the single-layered microcapsules in a polymer suspension produced in the first procedural step being pushed in turn through the inner channel of the three-channel spray nozzle, and at the same time a polymer solution without any biological cells being pushed through the outer channel of the three-channel spray nozzle, a chemically identical polymer or chemically different polymers in identical or different concentrations being used for the inner microcapsule and the at least one outer covering layer, it being possible moreover for the polymers to have different molar masses and/or different crosslinking.

2. The method according to claim 1 in which, instead of biological cells, tissue particles of human or animal origin are used, or biological cells and tissue particles are used at the same time.

3. The method according to claim 1, in which the diameter of the inner capsule is 10 to 2000 μm in size, and the outer covering layer has a thickness of from 10 to 2000 μm.

4. The method according to claim 1, in which several covering layers are applied and the inner covering layers contain biological cells and/or tissue particles which, if appropriate, can be different from the biological cells and/or tissue particles in the inner microcapsule.

5. The method according to claim 1, in which the multiple-layered microcapsules are enclosed with a polycation before or after application of the outer covering layer.

6. The method according to claim 5, the polycation being poly-L-lysine.

7. The method according to claim 1, instead of biological cells, substances being used.

8. The method according to claim 7, the substances being selected from therapeutic agents, cytostatic drugs and dietary supplements.

9. The method according to claim 8, the therapeutic agents being selected from proteins and hormones, the cytostatic drugs from proteins and angiogenesis inhibitors, and the dietary supplements being selected from vitamins and unsaturated fatty acids.

10. Spherical microcapsules with at least one outer covering layer made of a crosslinked polymer and a spherical core made of a crosslinked polymer and biological cells, obtained according to a method according to claim 1.

11. Use of the double- or multiple-layered microcapsules obtained according to a method according to claim 1, for producing a drug for transplant surgery.
